(19) United States
(12) Patent Application Publication
(10) Pub. No.: US 2004/0127893 A1
(43) Pub. Date: Jul. 1, 2004

(54) Methods for visualizing and treating intervertebral discs

(75) Inventor: David C. Hovda, Mountain View, CA (US)

Correspondence Address: ARTHROCARE CORPORATION, 680 VAQUEROS AVENUE, SUNNYVALE, CA, 94085-3523, US

(73) Assignee: ArthroCare Corporation, Sunnyvale, CA (US)

(21) Appl. No.: 10/735,477

(22) Filed: Dec. 11, 2003

Related U.S. Application Data
(60) Provisional application No. 60/433,250, filed on Dec. 13, 2002.

(51) Int. Cl. A61B 18/20; A61B 18/14

(52) U.S. Cl. 606/15; 606/41; 606/1

(57) ABSTRACT

Methods and systems for treating intervertebral discs and other difficult to access sites are provided. The methods include accessing the intervertebral disc with a small gauge access device and viewing a site within the disc with a fiber optic bundle delivered using the access device. The systems include a small gauge access device and a fiber optic bundle adapted to be delivered by means of the access device to a target tissue site.

### FIELD OF THE INVENTION

The present invention relates to visualization devices and methods for the treatment of intervertebral discs and other difficult to access target sites.

### BACKGROUND OF THE INVENTION

Intervertebral discs mainly function to articulate and cushion the vertebrae, while the interspinous tissue (i.e., tendons and cartilage, and the like) function to support the vertebrae so as to provide flexibility and stability to the patient's spine.

The discs comprise a nucleus pulposus which is a central hydrophilic cushion. The nucleus is surrounded by an annulus fibrosus or annulus which is a multi-layered fibrous ligament. The disc also includes vertebral endplates which are located between the disc and adjacent vertebrae.

The nucleus pulposus occupies 25-40% of the total disc cross-sectional area. It is composed mainly of mucoid material containing mainly proteoglycans with a small amount of collagen. The proteoglycans consist of a protein core having attached chains of negatively charged keratin sulphate and chondroitin sulphate. Such a structure is the reason the nucleus pulposus is a “loose or amorphous hydrogel” which has the capacity to bind water and usually contains 70-90% water by weight.

The annulus fibrosus forms the outer boundary of the disc and is composed of highly structured collagen fibers embedded in amorphous base substance also composed of water and proteoglycans. However, the amorphous base of the annulus is lower in content than in the nucleus. The collagen fibers of the annulus are arranged in concentric laminated bands. In each laminated band the fibers are parallel and attached to the adjacent vertebral bodies at roughly a 30° angle from the horizontal plane of the disc in both directions. There is a steady increase in the proportion of collagen from the inner to the outer annulus.

Each disc has two vertebral end-plates composed of hyaline cartilage. As mentioned above, the end-plates separate the disc from adjacent vertebral bodies. The end-plates act as a transitional zone between the harder bony vertebral bodies and the soft disc. Because the nucleus pulposus does not contain blood vessels (i.e., it is avascular), the disc receives most nutrients through the end-plate areas.

Many patients suffer from discogenic pain resulting from degenerative disc disease and/or vertebral disc herniation. Degeneration of discs occurs when they lose their water content and height, causing adjoining vertebrae to move closer together. The deterioration of the disc results in a decrease of the shock-absorbing ability of the spine. This condition also causes a narrowing of the neural openings in the sides of the spine which may pinch the spinal nerves. Thus disc degeneration may eventually cause severe chronic and disabling back and leg pain.

Disc herniations generally fall into three types of categories: 1) contained disc herniation (also known as contained disc protrusion); 2) extruded disc herniation; and 3) sequestered disc herniation (also known as a free fragment.)

In a contained herniation, a portion of the disc protrudes or bulges from a normal boundary of the disc. However, in a contained herniation, the nucleus pulposus does not breach the annulus fibrosus. Instead, a portion of the disc protrudes outwardly from an otherwise normal boundary of the disc. This protrusion might mechanically compress and/or chemically irritate an adjacent nerve root. This condition leads to radicular pain, commonly referred to as sciatica (leg pain.) In an extruded herniation, the annulus is disrupted and a segment of the nucleus protrudes/extrudes from the disc. However in this condition, the nucleus within the disc remains contiguous with the extruded fragment. With a sequestered disc herniation, a nucleus fragment separates from the nucleus and disc.

Degenerating or injured discs may have weaknesses in the annulus contributing to herniation of the disc. The weakened annulus may allow fragments of nucleus pulposus to migrate through the annulus fibrosus and into the spinal canal. Once in the canal, the displaced nucleus pulposus tissue, or the protruding annulus may impinge on spinal nerves or nerve roots. A weakened annulus may also result in bulging (e.g., a contained herniation) of the disc. Mechanical compression and/or chemical irritation of the nerve may occur depending on the proximity of the bulge to a nerve. A patient with these conditions may experience pain, sensory, and motor deficit.

A significant percentage of such patients undergo surgical procedures to treat the disorders described above. These procedures include both percutaneous and open discectomy, and spinal fusion.

Often, symptoms from disc herniation can be treated successfully by non-surgical means, such as rest, therapeutic exercise, oral anti-inflammatory medications or epidural injection of corticosteroids. Such treatments result in a gradual but progressive improvement in symptoms and allow the patient to avoid surgical intervention.

In some cases, the disc tissue is irreparably damaged, thereby necessitating removal of a portion of the disc or the entire disc to eliminate the source of inflammation and pressure. In more severe cases, the adjacent vertebral bodies must be stabilized following excision of the disc material to avoid recurrence of the disabling back pain. One approach to stabilizing the vertebrae, termed spinal fusion, is to insert an interbody graft or implant into the space vacated by the degenerative disc. In this procedure, a small amount of bone may be grafted and packed into the implants. This allows the bone to grow through and around the implant, fusing the vertebral bodies and preventing reoccurrence of the symptoms.

Open techniques for the treatment of herniated discs involve surgical dissection through soft tissue and removal of a portion of vertebral bone. Conventionally, upon encountering the annulus, a complex surgical incision, called an annulotomy, must be made to allow access of instruments into the disc. Mechanical instruments, such as pituitary rongeurs, curettes, graspers, cutters, drills, microdebriders and the like are often used to remove the nucleus material. Unfortunately, these mechanical instruments greatly lengthen and increase the complexity of the procedure. In addition, and most significantly, the annulotomy itself may lead to future re-herniation of the disc or even accelerate disc degeneration. Previously, in order to reduce the risk of re-herniation of the annulus subsequent to the performance of an annulotomy, the surgeon removes an excess amount of nucleus material from the disc than that required to normally decompress the disc. However, it was found that removing an excess amount of the nucleus pulposus destabilizes the disc leading to accelerated disc degeneration. See e.g., Meakin et al., The Effect of Partial Removal of the Nucleus Pulposus from the Intervertebral Disc on the Response of the Human Annulus Fibrosus to Compression, Clin Biomech (Bristol, Avon) 2001 February; 16(2) pp. 121-128. Discussions of the problems associated with annulotomy are found in journals and other medical publications. (See e.g., Ahlgren, et al. Annular incision technique on the strength and multidirectional flexibility of the healing intervertebral disc, Spine 1994, April 15; 9(8) pp 948-954; Ahlgren, et al. Effect of annular repair on the healing strength of the intervertebral disc: a sheep model, Spine 2000, September 1; 25(17): pp 2167-2170.)

Until recently, such surgical spinal procedures resulted in major operations and traumatic dissection of muscle and bone removal or bone fusion. However, the development of minimally invasive spine surgery instrumentation and percutaneous techniques has overcome many of the disadvantages of traditional traumatic spine surgery. Such percutaneous techniques for the treatment of herniated discs include: chemonucleolysis; laser techniques; and mechanical techniques, such as automated percutaneous lumbar discectomy. These procedures generally require the surgeon to place an introducer needle or cannula from the external surface of the patient to the spinal disc(s) for passage of surgical instruments or device. In percutaneous spinal procedures, the spinal canal is not violated and therefore epidural bleeding with ensuing scarring is minimized or completely avoided. In addition, the risk of instability from ligament and bone removal is generally lower in percutaneous procedures than with open procedures. Further, because percutaneous procedures involve much less trauma than open procedure, they facilitate a faster recovery and return to work by the patient.

Monopolar and bipolar radiofrequency devices have been used in limited roles in spine surgery. Such conventional RF devices have commonly known drawbacks. More recently, the application of Coblation® technology, as described herein, enabled numerous advantages when applied to vertebral disc procedures as compared to laser, mechanical, chemical, other RF devices, etc.

One such advantage is that the Coblation® technology volumetrically removes ablated tissue via molecular disintegration of larger organic molecules into smaller molecules and/or atoms, such as hydrogen, oxygen, oxides of carbon, hydrocarbons and nitrogen compounds. This molecular disintegration completely removes the ablated disc tissue, as opposed to dehydrating the tissue material by removing liquid from both within the tissue cells and extracellular fluids. Accordingly, Coblation® technology allows for a relatively small device (as compared to other treatment modalities) to enter through the annulus and remove the required amount of disc nucleus material. Thus, a smaller device minimizes the size of the incision that is made in the annulus or annular wall. Inventive methods, devices, and systems regarding this concept are discussed in more detail in commonly assigned provisional application No. 60/408,967, which is incorporated by reference.

Apart from the above, some medical practitioners may have a desire to actually observe/view/inspect the interior of a treated intervertebral disc either before or subsequent to any procedure. For example, a medical practitioners may seek to view the interior of a disc prior and/or subsequent to any procedure to internally inspect the disc as a cautionary measure or to inspect for various abnormalities (e.g., fissures, undesirable composition of the nucleus, etc.). Moreover, a medical practitioner may want to view the disc to determine a desirable a strategy for treatment. Such a desire to inspect the disc may be especially true for those practitioners who employ Coblation® technology since there is no solid tissue byproduct of any discarded/ablated disc-tissue as a result of the procedure.

Traditionally, medical practitioners employed endoscopes for visibility or enhanced visibility of the surgical site. Endoscopes are often used for minimally invasive procedures (as well as certain open procedures). As an example, there are two common categories of endoscopes intended for use in such spinal procedures.

The first common category of endoscope is a stand-alone scope that provides only a viewing/visualization means that is deliverable to an access site via a hollow needle or cannula. Because such hollow needle or cannula is often used to deliver other instruments, e.g., cutting instruments to effect the annulotomy, its diameter is relatively large. Alternatively, more than one cannula is used—one designated for delivery of the scope and another for delivery of working instrumentation. One such multi-cannulae system is described in U.S. Pat. 5,762,629.

The second common category of endoscopes is one which integrates other components and channels within a single device. For example, the Yeung Endoscopic Spine Surgery System (YESS), made by the Richard Wolf Medical Instruments Corp. of Vernon Hills, Ill., includes a multi-channel endoscope with a viewing means, a working channel through which instruments are delivered to the surgical site, irrigation inflow and outflow channels, and a laser fiber channel.

However, the approaches described above which employ traditional endoscopes in spinal surgery are less than optimal. For example, such approaches may be suited to prior surgical techniques where it was acceptable for the practitioner to only view the exterior of a disc.

Thus, there remains a need to image/view/inspect/etc. the interior of a vertebral disc either prior to or subsequent to treatment of the disc. Such a need is made more evident given a need to minimize the entry point into the annulus and also by the increased use of Coblation® technology to treat vertebral discs.

### SUMMARY OF THE INVENTION

It is understood that while the present invention is suited for use with Coblation® based treatment of the vertebral disc, it is not limited as such. The present invention may be employed with any known treatment modality for vertebral discs.

The invention includes a method for treating an intervertebral disc comprising advancing at least one optic fiber into a nucleus of the disc via the access device and viewing an interior of the disc using at least one of the optic fibers.

In one variation, the invention further includes advancing an access device into the disc to create a passageway into the disc with the access device. Advancing the access device into the disc may include separating layers of a fibrous outer portion of the disc to create a passageway into the disc with the access device.

Another variation of the invention may further include advancing a treatment device through the access device, and activating the treatment device to treat the disc. The treatment device may be activated prior to or subsequent to viewing the interior of the disc. Moreover, advancing of the optic fiber and viewing the interior of the disc are performed intermittently throughout the method.

In another variation of the invention, advancing the access device may include inserting a needle into at least a fibrous outer portion of the disc.

Variations of treatment devices for use with the present invention may include, but are not limited to pituitary rongeurs, curettes, graspers, cutters, drills, and microdebriders. Furthermore, a treatment device comprising an electrosurgical device may be used in combination with the inventive method.

The optic fiber of the present device may be used to observe the effect of treatment on an outer portion of the disc or an interior of the discs.

The invention also includes kits for treating a vertebral disc comprising. The kits may comprise a treatment device and at least one optic fiber or optic bundle.

The treatment device of the kit may be one or more of those described herein.

### BRIEF DESCRIPTION OF THE DRAWINGS

FIG. 1 is a top view of an access device of the present invention which has been penetrated into a target disc for treatment of that disc.

FIG. 1A is a view of the access device of FIG. 1 along the lines A-A.

FIG. 2 illustrates the removal of a stylet from the access device of FIG. 1.

FIG. 3. illustrates the insertion of an optic fiber bundle into the access device of FIG. 2.

FIG. 4 illustrates an exemplary treatment device according to a variation of the present invention.

FIG. 5 is an enlarged view of the target disc having received within the access device an RF treatment device of the present invention.

FIG. 6 is an enlarged view of the target disc illustrating the resulting effect after various applications of the RF treatment device within the disc.

FIG. 7 is an enlarged view of the target disc subsequent to removal of the treatment and access devices.

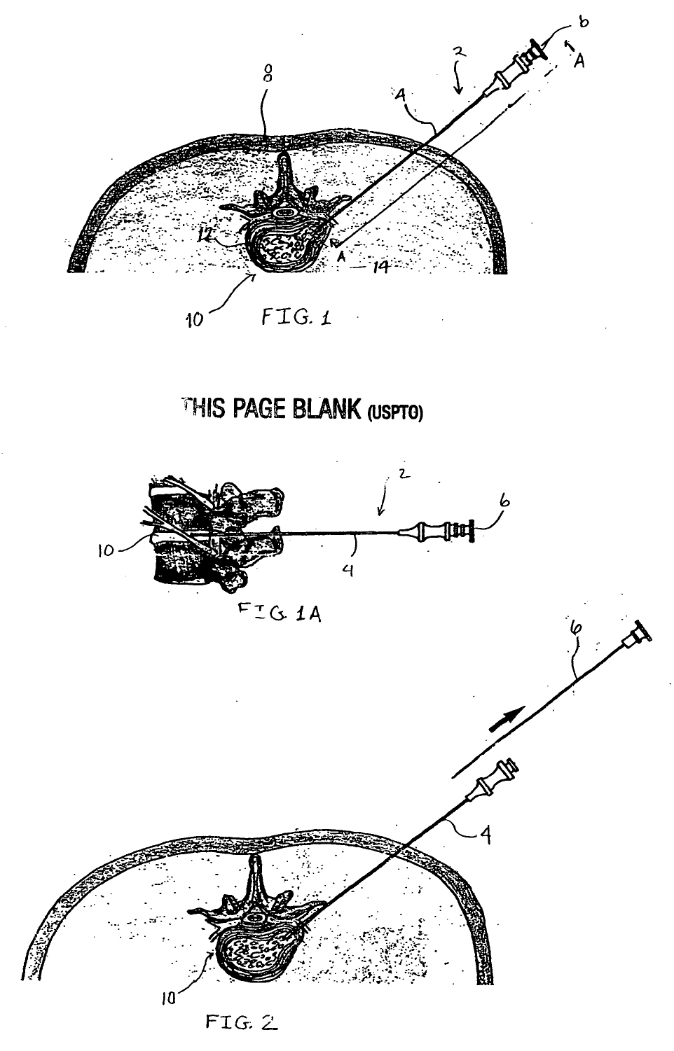

### DESCRIPTION OF A PREFERRED EMBODIMENT OF THE PRESENT INVENTION

Before the present invention is described in detail, it is to be understood that this invention is not limited to particular variations set forth herein as various changes or modifications may be made to the invention described and equivalents may be substituted without departing from the true spirit and scope of the invention. As will be apparent to those of skill in the art upon reading this disclosure, each of the individual embodiments described and illustrated herein has discrete components and features which may be readily separated from or combined with the features of any of the other several embodiments without departing from the scope or spirit of the present invention. In addition, many modifications may be made to adapt a particular situation, material, composition of matter, process, process act(s) or step(s) to the objective(s), spirit or scope of the present invention. All such modifications are intended to be within the scope of the claims made herein.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

All existing subject matter mentioned herein (e.g., publications, patents, patent applications and hardware) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Last, it is to be appreciated that unless defined otherwise, all technical and scientific terms used herein have the same meaning as commonly understood by one of ordinary skill in the art to which this invention belongs.

The systems of the present invention may be configured to address any application wherein a viewing means is needed to visualize a target site which is not readily visible to the physician performing the procedure. The subject systems are particularly suitable for treating all types of intervertebral discs, including degenerative discs, at all levels of the vertebral column. The subject systems include at least an access device and a viewing means which is deliverable through the access device. In certain embodiments, the system further includes a treatment device suitably configured for treating the degenerative intervertebral disc.

In one variation, an optic fiber is used as a viewing means for the present invention. It is contemplated that the term optic fiber includes: a single fiber optic probe; a single optic fiber; an optic cable; and/or a bundle of optic fibers, probes, or cables. The optic fiber may be configured to have a diameter that accommodates the hollow needle or thin-walled cannula used to enter the annulus of the disc. For example, the diameter of the fiber, or fiber bundle, may be the same as a 26GA needle (0.018″). Alternatively, the fiber, or fiber bundle, may be as large as required (e.g., a diameter of a 16-17 GA needle ,0.065″-0.058″). However, the invention includes fibers and fiber bundles of any size as required.

The viewing means may be provided with an eye piece for line of sight viewing by the physician or may be connectable to a video monitor. It is contemplated that the present invention may use viewing measures other than optic fibers, e.g., cameras, CCD device, diodes, etc. Furthermore, it is also contemplated that the viewing measures, optic fibers, etc. as discussed herein may be incorporated on or in the treatment device.

The treatment device of the present invention may have a variety of configurations as described above. However, one variation of the invention employs a treatment device using Coblation® technology.

As stated above, the assignee of the present invention developed Coblation® technology. Coblation® technology involves the application of a high frequency voltage difference between one or more active electrode(s) and one or more return electrode(s) to develop high electric field intensities in the vicinity of the target tissue. The high electric field intensities may be generated by applying a high frequency voltage that is sufficient to vaporize an electrically conductive fluid over at least a portion of the active electrode(s) in the region between the distal tip of the active electrode(s) and the target tissue. The electrically conductive fluid may be a liquid or gas, such as isotonic saline, blood, extracelluar or intracellular fluid, delivered to, or already present at, the target site, or a viscous fluid, such as a gel, applied to the target site.

When the conductive fluid is heated enough such that atoms vaporize off the surface faster than they recondense, a gas is formed. When the gas is sufficiently heated such that the atoms collide with each other causing a release of electrons in the process, an ionized gas or plasma is formed (the so-called “fourth state of matter”). Generally speaking, plasmas may be formed by heating a gas and ionizing the gas by driving an electric current through it, or by shining radio waves into the gas. These methods of plasma formation give energy to free electrons in the plasma directly, and then electron-atom collisions liberate more electrons, and the process cascades until the desired degree of ionization is achieved. A more complete description of plasma can be found in Plasma Physics, by R. J. Goldston and P. H. Rutherford of the Plasma Physics Laboratory of Princeton University (1995), the complete disclosure of which is incorporated herein by reference.

As the density of the plasma or vapor layer becomes sufficiently low (i.e., less than approximately 1020 atoms/cm3 for aqueous solutions), the electron mean free path increases to enable subsequently injected electrons to cause impact ionization within the vapor layer). Once the ionic particles in the plasma layer have sufficient energy, they accelerate towards the target tissue. Energy evolved by the energetic electrons (e.g., 3.5 eV to 5 eV) can subsequently bombard a molecule and break its bonds, dissociating a molecule into free radicals, which then combine into final gaseous or liquid species. Often, the electrons carry the electrical current or absorb the radio waves and, therefore, are hotter than the ions. Thus, the electrons, which are carried away from the tissue towards the return electrode, carry most of the plasma's heat with them, allowing the ions to break apart the tissue molecules in a substantially non-thermal manner.

By means of this molecular dissociation (rather than thermal evaporation or carbonization), the target tissue structure is volumetrically removed through molecular disintegration of larger organic molecules into smaller molecules and/or atoms, such as hydrogen, oxygen, oxides of carbon, hydrocarbons and nitrogen compounds. This molecular disintegration completely removes the tissue structure, as opposed to dehydrating the tissue material by the removal of liquid within the cells of the tissue and extracellular fluids, as is typically the case with electrosurgical desiccation and vaporization. A more detailed description of this phenomena can be found in commonly assigned U.S. Pat. No. 5,697,882 the complete disclosure of which is incorporated herein by reference.

In some applications of the Coblation® technology, high frequency (RF) electrical energy is applied in an electrically conducting media environment to shrink or remove (i.e., resect, cut, or ablate) a tissue structure and to seal transected vessels within the region of the target tissue. Coblation® technology is also useful for sealing larger arterial vessels, e.g., on the order of about 1 mm in diameter. In such applications, a high frequency power supply is provided having an ablation mode, wherein a first voltage is applied to an active electrode sufficient to effect molecular dissociation or disintegration of the tissue, and a coagulation mode, wherein a second, lower voltage is applied to an active electrode (either the same or a different electrode) sufficient to heat, shrink, and/or achieve hemostasis of severed vessels within the tissue.

The amount of energy produced by the Coblation® device may be varied by adjusting a variety of factors, such as: the number of active electrodes; electrode size and spacing; electrode surface area; asperities and sharp edges on the electrode surfaces; electrode materials; applied voltage and power; current limiting means, such as inductors; electrical conductivity of the fluid in contact with the electrodes; density of the fluid; and other factors. Accordingly, these factors can be manipulated to control the energy level of the excited electrons. Since different tissue structures have different molecular bonds, the Coblation® device may be configured to produce energy sufficient to break the molecular bonds of certain tissue but insufficient to break the molecular bonds of other tissue. For example, fatty tissue (e.g., adipose) has double bonds that require an energy level substantially higher than 4 eV to 5 eV (typically on the order of about 8 eV) to break. Accordingly, the Coblation® technology generally does not ablate or remove such fatty tissue; however, it may be used to effectively ablate cells to release the inner fat content in a liquid form. Of course, factors may be changed such that these double bonds can also be broken in a similar fashion as the single bonds (e.g., increasing voltage or changing the electrode configuration to increase the current density at the electrode tips). A more complete description of this phenomena can be found in commonly assigned U.S. Pat. Nos. 6,355,032, 6,149,120 and 6,296,136, the complete disclosures of which are incorporated herein by reference.

The active electrode(s) of a Coblation® device may be supported within or by an inorganic insulating support positioned near the distal end of the instrument shaft. The return electrode may be located on the instrument shaft, on another instrument or on the external surface of the patient (i.e., a dispersive pad). The proximal end of the instrument(s) will include the appropriate electrical connections for coupling the return electrode(s) and the active electrode(s) to a high frequency power supply, such as an electrosurgical generator.

A more detailed discussion of applications and devices using Coblation(® technology as applied to intervertebral discs may be found as follows. Issued U.S. Pat. Nos. 6,283,961; 6,264,651; 6,277,112; 6,322,549; 6,045,532; 6,264,650; 6,464,695; 6,468,274; and 6,468,270 each of which is incorporated by reference. Pending U.S. applications Ser. No. 09/676,194 filed Sep. 28, 2000; 09/679,394 filed Oct. 3, 2000; 09/747,311 filed Dec. 20, 2000; 09/665,441 filed Sep. 19, 2000; 09/765,832 filed Jan. 19, 2001; and 09/848,843 filed May 3, 2001, each of which is incorporated by reference. Provisional application No. 60/408,967, which is incorporated by reference.

In one example of a Coblation® device for use with the present invention, the return electrode of the device is typically spaced proximally from the active electrode(s) a suitable distance to avoid electrical shorting between the active and return electrodes in the presence of electrically conductive fluid. In many cases, the distal edge of the exposed surface of the return electrode is spaced about 0.5 mm to 25 mm from the proximal edge of the exposed surface of the active electrode(s), preferably about 1.0 mm to 5.0 mm. Of course, this distance may vary with different voltage ranges, conductive fluids, and depending on the proximity of tissue structures to active and return electrodes. The return electrode will typically have an exposed length in the range of about 1 mm to 20 mm.

A Coblation® treatment device for use in the present invention may use a single active electrode or an array of active electrodes spaced around the distal surface of a catheter or probe. In the latter embodiment, the electrode array usually includes a plurality of independently current-limited and/or power-controlled active electrodes to apply electrical energy selectively to the target tissue while limiting the unwanted application of electrical energy to the surrounding tissue and environment resulting from power dissipation into surrounding electrically conductive fluids, such as blood, normal saline, and the like. The active electrodes may be independently current-limited by isolating the terminals from each other and connecting each terminal to a separate power source that is isolated from the other active electrodes. Alternatively, the active electrodes may be connected to each other at either the proximal or distal ends of the catheter to form a single wire that couples to a power source.

In one configuration, each individual active electrode in the electrode array is electrically insulated from all other active electrodes in the array within the instrument and is connected to a power source which is isolated from each of the other active electrodes in the array or to circuitry which limits or interrupts current flow to the active electrode when low resistivity material (e.g., blood, electrically conductive saline irrigant or electrically conductive gel) causes a lower impedance path between the return electrode and the individual active electrode. The isolated power sources for each individual active electrode may be separate power supply circuits having internal impedance characteristics which limit power to the associated active electrode when a low impedance return path is encountered. By way of example, the isolated power source may be a user selectable constant current source. In this embodiment, lower impedance paths will automatically result in lower resistive heating levels since the heating is proportional to the square of the operating current times the impedance. Alternatively, a single power source may be connected to each of the active electrodes through independently actuatable switches, or by independent current limiting elements, such as inductors, capacitors, resistors and/or combinations thereof. The current limiting elements may be provided in the instrument, connectors, cable, controller, or along the conductive path from the controller to the distal tip of the instrument. Alternatively, the resistance and/or capacitance may occur on the surface of the active electrode(s) due to oxide layers which form selected active electrodes (e.g., titanium or a resistive coating on the surface of metal, such as platinum).

The Coblation® device is not limited to electrically isolated active electrodes, or even to a plurality of active electrodes. For example, the array of active electrodes may be connected to a single lead that extends through the catheter shaft to a power source of high frequency current.

The voltage difference applied between the return electrode(s) and the active electrode(s) will be at high or radio frequency, typically between about 5 kHz and 20 MHz, usually being between about 30 kHz and 2.5 MHz, preferably being between about 50 kHz and 500 kHz, often less than 350 kHz, and often between about 100 kHz and 200 kHz. In some applications, applicant has found that a frequency of about 100 kHz is useful because the tissue impedance is much greater at this frequency. In other applications, such as procedures in or around the heart or head and neck, higher frequencies may be desirable (e.g., 400-600 kHz) to minimize low frequency current flow into the heart or the nerves of the head and neck.

The RMS (root mean square) voltage applied will usually be in the range from about 5 volts to 1000 volts, preferably being in the range from about 10 volts to 500 volts, often between about 150 volts to 400 volts depending on the active electrode size, the operating frequency and the operation mode of the particular procedure or desired effect on the tissue (i.e., contraction, coagulation, cutting or ablation.)

Typically, the peak-to-peak voltage for ablation or cutting with a square wave form will be in the range of 10 volts to 2000 volts and preferably in the range of 100 volts to 1800 volts and more preferably in the range of about 300 volts to 1500 volts, often in the range of about 300 volts to 800 volts peak to peak (again, depending on the electrode size, number of electrons, the operating frequency and the operation mode). Lower peak-to-peak voltages will be used for tissue coagulation, thermal heating of tissue, or collagen contraction and will typically be in the range from 50 to 1500, preferably 100 to 1000 and more preferably 120 to 400 volts peak-to-peak (again, these values are computed using a square wave form). Higher peak-to-peak voltages, e.g., greater than about 800 volts peak-to-peak, may be desirable for ablation of harder material, such as bone, depending on other factors, such as the electrode geometries and the composition of the conductive fluid.

As discussed above, the voltage is usually delivered in a series of voltage pulses or alternating current of time varying voltage amplitude with a sufficiently high frequency (e.g., on the order of 5 kHz to 20 MHz) such that the voltage is effectively applied continuously (as compared with, e.g., lasers claiming small depths of necrosis, which are generally pulsed about 10 Hz to 20 Hz). In addition, the duty cycle (i.e., cumulative time in any one-second interval that energy is applied) is on the order of about 50% for the present invention, as compared with pulsed lasers which typically have a duty cycle of about 0.0001%.

The preferred power source of the present invention delivers a high frequency current selectable to generate average power levels ranging from several milliwatts to tens of watts per electrode, depending on the volume of target tissue being treated, and/or the maximum allowed temperature selected for the instrument tip. The power source allows the user to select the voltage level according to the specific requirements of a particular neurosurgery procedure, cardiac surgery, arthroscopic surgery, dermatological procedure, ophthalmic procedures, open surgery or other endoscopic surgery procedure. For cardiac procedures and potentially for neurosurgery, the power source may have an additional filter, for filtering leakage voltages at frequencies below 100 kHz, particularly voltages around 60 kHz. Alternatively, a power source having a higher operating frequency, e.g., 300 kHz to 600 kHz may be used in certain procedures in which stray low frequency currents may be problematic. A description of one suitable power source can be found in commonly assigned U.S. Pat. Nos. 6,142,992 and 6,235,020, the complete disclosure of both patents are incorporated herein by reference for all purposes.

The power source may be current limited or otherwise controlled so that undesired heating of the target tissue or surrounding (non-target) tissue does not occur. In a presently preferred embodiment of the present invention, current limiting inductors are placed in series with each independent active electrode, where the inductance of the inductor is in the range of 10 uH to 50,000 uH, depending on the electrical properties of the target tissue, the desired tissue heating rate and the operating frequency. Alternatively, capacitor-inductor (LC) circuit structures may be employed, as described previously in U.S. Pat. No. 5,697,909, the complete disclosure of which is incorporated herein by reference. Additionally, current-limiting resistors may be selected. Preferably, these resistors will have a large positive temperature coefficient of resistance so that, as the current level begins to rise for any individual active electrode in contact with a low resistance medium (e.g., saline irrigant or blood), the resistance of the current limiting resistor increases significantly, thereby minimizing the power delivery from said active electrode into the low resistance medium (e.g., saline irrigant or blood).

The following discussion is an example of the inventive method as applied to a percutaneous intervertebral disc-procedure using Coblation® technology. It is understood that the invention is not limited to a percutaneous procedure. Instead, the use of an optic fiber to view the interior of a disc may be applied in an open-surgical vertebral disc-procedure as well. In such a case, the optic fiber may be advanced to the disc using an endoscope, other introducer device, or by itself. Moreover, other treatment modalities (e.g., laser, chemical, other RF devices, etc.) may be used in the inventive method either in place of the Coblation® technology or in addition thereto.

To access a herniated disc in a percutaneous procedure, an access device is used, usually via fluoroscopy, to approach the disc. The access device 2, may be a hollow needle or thin-walled cannula 4 and usually includes a stylet 6 when advanced to the disc.

As discussed above, the annulus fibrosus 12 forms the outer boundary of the disc and is composed of highly structured collagen fibers embedded in amorphous base substance also composed of water and proteoglycans. However, the amorphous base of the annulus 12 comprises collagen fibers that are arranged in concentric laminated bands.

In one variation of the invention, there is a desire to minimize the trauma to the annulus of the vertebral disc. To accomplish this task, the opening created by the needle 4 into the annulus 12 is minimal and does not rely on removing a significant amount of annulus 12 material. As the needle 4 advances into the annulus 12, it separates the bands of the annulus. It should be noted that the needle 4 may have a sharp tip with a taper that serves to separate the laminated bands, or the needle may be blunt whereby the laminated bands are separated via blunt dissection. As a result, when the devices are subsequently removed from the annulus 12, the laminated bands relax independently of one another and re-orient. This re-orientation of the laminated bands closes the passageway created by the needle because the passageway formed by the separated bands is no longer in alignment. Essentially, individual movement of the bands dissipates the opening. Heat may be applied either prior to or subsequent to insertion of the needle 4 or even after removal of the needle 12. The heat is intended to cause shrinkage of the collagen fibers to increase movement of the fibers to aid in closure of the opening.

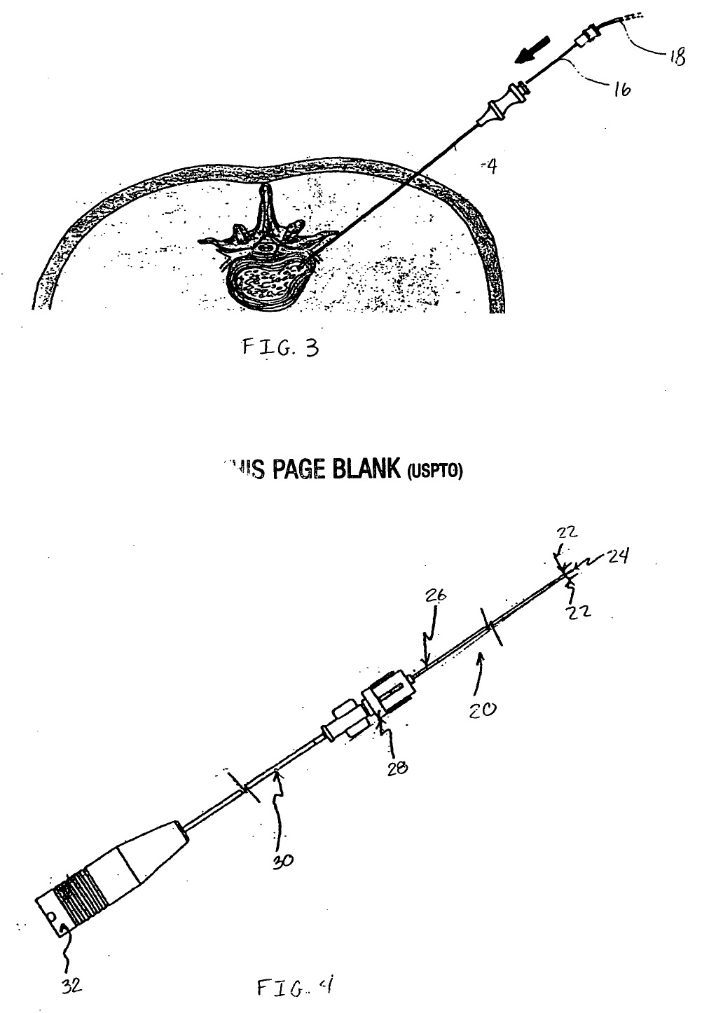

Next, as illustrated in FIG. 2, a stylet 6, if used, is removed from needle 4, and as shown in FIG. 3, a fiber optic bundle 16 as described above is inserted into needle 4 until the distal tip is positioned at the distal edge of needle 4. The targeted access site is then illuminated either by a different light source, or the same fiber or optical bundle, and then visualized via the optical fibers to verify the location of the damage or herniation and to assess the extent of such damage or herniation. As mentioned above, fiber optic bundle 16 may extend via cable 18 to an eye piece or to a video monitor. After the physician is satisfied with his visual assessment of the target site, the fiber optic bundle 16 is removed from needle 4 and a treatment device, such as the treatment device 20 of FIG. 4, is inserted into needle 4.

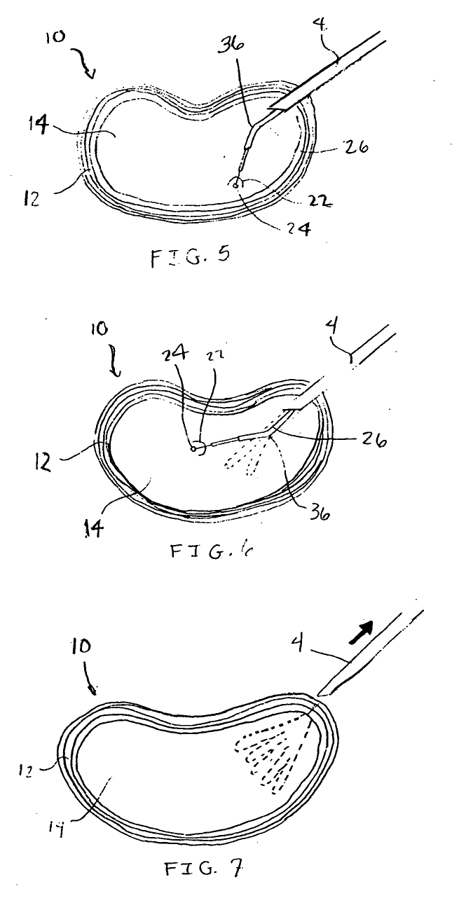

A treatment device or probe 20 is then inserted into the disc. It is noted that the invention is not limited to the treatment device show. The treatment device has electrodes 22 and 24 provided at the distal end of the shaft and may use bodily fluids to provide a conductive path between the electrodes. Alternatively, a conductive medium may be applied to the probe 20 or to the disc. In the variation shown in FIGS. 5 and 6, electrodes 22 and 24 are extendable from shaft 26 which may be bent or biased at a distal end to provide an angled or curved tip 36. As such, the ablation path of probe 20 is a curved trajectory. Alternatively, the shaft may be straight. Or, the probe may have a single electrode, or electrodes that do not extend from the shaft. In other variations, the distal portion of shaft of the probe 20 comprises a flexible material which can be deflected relative to the longitudinal axis of the shaft. Such deflection may be selectively induced by mechanical tension of a pull wire, for example, or by a shape memory wire that expands or contracts by externally applied temperature changes. Moreover, a curved introducer needle 4 may be used to provide a curved trajectory. A more complete description of this embodiment can be found in U.S. Pat. No.5,697,909, the complete disclosure of which has previously been incorporated herein by reference. In additional variations, the needle 4 or probe 20 may be bent by the physician to the appropriate angle using a conventional bending tool or the like.

The proximal end of the shaft 26 may include one or more handles or hubs 28. Hub 28 may connect to an extension cable or “pig-tail” 30 which is ultimately connected to a handle 32 that is adapted to couple to a power supply (not shown). The hub 28 may be a separate catheter, such as a commonly known break-away-introducer which assists the medical practitioner in placing the device. Or, the hub 28 may be affixed to the shaft 26 and/or pig-tail 30 thereby assisting the medical practitioner in positioning of the device. Furthermore, the hub 28 may incorporate features for use in determining the travel distance within the body (e.g., functioning as a stop-mechanism).

Alternatively, the probe may be configured with an integral cable that couples to a power supply.

FIG. 5 illustrates advancement of probe 20 into the nucleus pulposus 14 of the disc 10. While the inventive procedure is not limited to any particular electrosurgical probe, probe 20 may be inserted into the disc and, once inside, the probe 20 may ablate tissue as it is advanced through the nucleus of the disc.

Regardless of the type of treatment device employed, the device is operatively used to debulk the disc from impinging on a nerve root, or to treat fissures within the annulus wall.

Depending on the procedure, the surgeon may translate or otherwise move the electrodes relative to the target disc tissue to form one or more voids, holes, channels, stripes, divots, craters, or the like within the disc, as shown in FIG. 6. In addition, the surgeon may purposely create some thermal damage within these holes, or channels to form scar tissue that will stiffen and debulk the disc. In one variation, the physician axially translates the electrode assembly into the disc tissue as the tissue is volumetrically removed to form one or more channels therein. The channels will typically have a diameter of less than 15 mm, preferably around 5 mm. Applicant has found that probe 20 can quickly and cleanly create such holes, divots, or channels in tissue with the ablation technology described herein. A more complete description of methods for forming holes or channels in tissue can be found in U.S. Pat. No. 5,683,366, the complete disclosure of which is incorporated herein by reference for all purposes.

Throughout the treatment procedure, the physician may at any time interchange probe 20 with optical fiber bundle 16 to assess the ablated or shrunken tissue, and the status of the tissue and to determine where to apply the next treatment within the nucleus. The physician may then withdraw the optic fiber from the annulus to externally view the disc to determine whether sufficient debulking of the vertebral disc occurred. After sufficient ablation is applied, the treatment and access devices are removed from the disc, as illustrate in FIG. 7 and the herniation of the disc 10 is reduced or eliminated. The laminated bands within the annulus 12 relax and begin to close the opening made by needle 4. As described above, heat may be applied to the area of the opening to aid in closure.

The invention herein is described by examples and a desired way of practicing the invention is described. However, the invention as claimed herein is not limited to that specific description in any manner. Equivalence to the description as hereinafter claimed is considered to be within the scope of protection of this patent.

What is claimed is:

1. A method for treating an intervertebral disc comprising:
advancing at least one optic fiber into a nucleus of the disc via the access device; and
viewing an interior of the disc using at least one of the optic fibers.

2. The method of claim 1 further comprising advancing an access device into the disc to create a passageway into the disc with the access device.

3. The method of claim 2 where advancing the access device into the disc comprises separating layers of a fibrous outer portion of the disc to create a passageway into the disc with the access device.

4. The method of claim 2 further comprising;
advancing a treatment device through the access device; and
activating the treatment device to treat the disc.

5. The method of claim 4, wherein activating the treatment device occurs prior to viewing the interior of the disc.

6. The method of claim 4, wherein at least one of the optic fibers is attached to the treatment device.

7. The method of claim 4, wherein said advancing of the at least one optic fiber and viewing the interior of the disc are performed intermittently throughout said method.

8. The method of claim 2, wherein advancing the access device comprises inserting a needle into at least a fibrous outer portion of the disc.

9. The method of claim 2 where the treatment device is selected from a group comprising pituitary rongeurs, curettes, graspers, cutters, drills, and microdebriders.

10. The method of claim 4, wherein the treatment device includes at least one active electrode and a return electrode, wherein activating the treatment device comprises applying a high frequency voltage between the active and return electrodes.

11. The method of claim 10, further comprises using a conductive medium to form a current path between the active and return electrodes.

12. The method of claim 11, where the conductive medium is provided from a source external to the disc.

13. The method of claim 11, where the conductive medium is the naturally occurring fluid within the disc.

14. The method of claim 2, wherein advancing the treatment device comprises advancing the treatment device into a nucleus pulposus of the disc.

15. The method of claim 4, wherein activating the treatment device comprises ablating tissue within the disc.

16. The method of claim 15, further comprising observing the effect of the ablating of tissue using the optic fiber.

17. The method of claim 16, wherein observing the effect comprises measuring a void created by the ablating of tissue.

18. The method of claim 16, wherein observing the effect comprises observing an outer portion of the disc.

19. The method of claim 4, wherein activating the treatment device comprises coagulating tissue within the disc.

20. The method of claim 19, further comprising observing the effect of the coagulating of tissue using the optic fiber.

21. The method of claim 20, wherein observing the effect comprises measuring shrinkage of tissue resulting from the coagulating of tissue.

22. The method of claim 20, wherein observing the effect comprises observing an outer portion of the disc.

23. The method of claim 4, further comprising performing non-invasive imaging prior to or during activating the treatment device.

24. The method of claim 1, further comprising injecting a dye into the disc.

25. The method of claim 1, where advancing the at least one optic fiber into the nucleus of the disc via the access device is performed during an open surgical procedure.

26. The method of claim 1, where advancing the at least one optic fiber into the nucleus of the disc via the access device is performed during a percutaneous surgical procedure.
